(19) United States
(12) Patent Application Publication
(10) Pub. No.: US 2025/0288751 A1
(43) Pub. Date: Sep. 18, 2025

(54) DEVICES AND METHODS FOR SIMULTANEOUS MULTISITE INJECTION

(71) Applicant: ONEPASS MEDICAL LTD., Katzrin (IL)

(72) Inventors: Jacob BEN ARIE, Haifa (IL); Jannette LAZAROVITS, Reut (IL); Yaron AMIKAM, Carmiel (IL); Dimitri DUKHNO, Kiryat Motzkin (IL)

(73) Assignee: ONEPASS MEDICAL LTD., Katzrin (IL)

(21) Appl. No.: 18/862,614

(22) PCT Filed: May 1, 2023
(86) PCT No.: PCT/IL2023/050444

§ 371 Date: Nov. 4, 2024

Related U.S. Application Data
(60) Provisional application No. 63/337,315, filed on May 2, 2022.

(51) Int. Cl. A61M 5/32; A61M 5/315; A61M 25/00

(52) U.S. Cl. CPC A61M 5/3298; A61M 5/31578; A61M 25/0084

(57) ABSTRACT

A device for multisite injection is disclosed. The device comprises, a first container in fluid connection with at least two needles; a second container in fluid connection with at least one needle; and an injection unit configured to inject an injection fluid from the first container and the second container via all the needles.

### CROSS-REFERENCE TO RELATED APPLICATION

This application claims the benefit of priority of U.S. Provisional Patent Application No. 63/337,315, titled “DEVICES AND METHODS FOR SIMULTANEOUS MULTISITE INJECTION”, filed May 2, 2022, the contents of which are incorporated herein by reference in their entirety.

### FIELD OF THE INVENTION

The present invention relates generally to devices and methods for medication injection. More specifically, the present invention relates devices and methods for simultaneous multisite injection.

### BACKGROUND OF THE INVENTION

Multisite injection of active materials, for example, medications or other active fluid was proven to be more effective than a single injection. For example, multisite injection therapy was found to be more effective for shoulder injury therapy when comparing the pain relief, range of motion (ROM), and functional scores of randomly selected patients with primary frozen shoulder. In yet another example, in direct cancer treatment, a multisite injection of the tumor was found to be more effective than providing the entire dose in a single injection.

The current multisite injection technique involves using a single needle for conducting all the injections, re-puncturing the tissue in each injection separately, and relying on the physician's technique to provide the accurate amount of medication each time.

Accordingly, there is a need for a device that will allow a simple multisite injection of a controlled amount of fluid, while ensuring effective dispersion of the injected fluid.

### SUMMARY OF THE INVENTION

Some aspects of the invention are directed to a device for multisite injection, comprising: a first container in fluid connection with at least two needles; a second container in fluid connection with at least one needle; and an injection unit configured to inject an injection fluid from the first container and the second container via all the needles.

In some embodiments, the injection unit is configured to inject the injection fluid simultaneously via all the needles. In some embodiments, the injection unit is configured to inject the injection fluid sequentially by at least two needles. In some embodiments, the injection unit is configured to inject a controlled amount of injection fluid from each needle. In some embodiments, the controlled amount is the same amount from at least two needles.

In some embodiments, the device further comprises a catheter comprising a plurality of lumens, each being in fluid connection with one needle. In some embodiments, the device further comprises a supporting splitter located at a distal end of the catheter opposite to the injection unit, wherein the supporting splitter comprises a plurality of conduits for directing each one of the needles.

In some embodiments, the first container and the at least two needles are connected via a divider configured to divide the injection fluid between the at least two needles. In some embodiments, the divider is included in a catheter. In some embodiments, the divider comprises a main lumen split into two or more sub-lumens, wherein each sub-lumen is connectable to one needle.

In some embodiments, the first container is configured to hold a first amount of injection fluid and the second container is configured to hold a second amount of injection fluid different from the first. In some embodiments, the first container and the second container are syringe barrels and wherein the injection unit is configured to control the movement of a plunger located in each syringe barrel. In some embodiments, the injection unit comprises at least one pump connected to at least one of the first container and the second container.

In some embodiments, the device further comprises a controller configured to control the injection unit to simultaneously extract the injection fluid from the first container and the second container.

An additional aspect of the invention may be directed to: a device for multisite injection, comprising: a support holding: at least a first needle in fluid connection to a first lumen prefilled with a first injection fluid; and at least a second needle in fluid connection to a second lumen prefilled with a second injection fluid; and an injection unit configured to inject the first injection fluid and the second injection fluid via the corresponding needle.

In some embodiments, the first injection fluid and the second injection fluid are the same fluid. In some embodiments, the first injection fluid is provided in a first amount and the second injection fluid is provided in a second amount. In some embodiments, the first amount and the second amount are the same.

In some embodiments, the injection unit is configured to inject the injection fluid simultaneously via all the needles. In some embodiments, the injection unit is configured to inject the injection fluid sequentially by at least two needles. In some embodiments, the device further comprises a catheter for holding at least one needle. In some embodiments, the injection unit comprises at least one plunger configured to apply pressure on the first injection fluid in the first lumen and on the second injection fluid in the second lumen.

Some additional aspects of the invention are directed to a method of multisite injection, comprising: providing a device for multisite injection, comprising: a first container in fluid connection with at least two needles; a second container in fluid connection with at least one needle; and an injection unit configured to inject an injection fluid from the first container and the second container via all the needles; loading the first container and the second container with an injection fluid; and injecting the injection fluid to multiple sites in a tissue via all the needles.

In some embodiments, injecting the injection fluid is conducted situationally from all needles. In some embodiments, injecting the injection fluid is conducted sequentially by at least two needles. In some embodiments, injecting the injection fluid comprises injecting the same volume of injection fluid via at least two of the needles.

Some additional aspects of the invention are directed to another method of multisite injection, comprising: providing a device for multisite injection, comprising: a housing holding: at least a first needle in fluid connection to a first lumen/channel prefilled with a first injection fluid; and at least a second needle in fluid connection to a second lumen/channel prefilled with a second injection fluid; and an injection unit configured to inject the first injection fluid and the second injection fluid via the corresponding needle and injecting the first injection fluid and the second injection fluid via all needles.

In some embodiments, injecting the first injection fluid and the second injection fluid is conducted situationally from all needles. In some embodiments, injecting the second injection fluid is conducted sequentially after the injection of the second injection fluid. In some embodiments, the first injection fluid and the second injection fluid are the same.

Some additional aspects of the invention are directed to a supporting splitter for supporting needles, comprising: a body insertable into a catheter holding the needles, comprising; at least one first conduit for supporting at least one first needle; and at least one second conduit for supporting at least one second needle, such that the at least one first needle is spread from the at least one second needle.

In some embodiments, the first conduit is a first lumen and the second conduit is a second lumen. In some embodiments, the supporting splitter comprises a plurality of first and second conduits each shaped as a lumen, circumferentially located near the walls of the body. In some embodiments, the supporting splitter comprises a plurality of first lumens circumferentially located near the walls of the body and wherein the second conduit is a second lumen located along the longitudinal central axis of the body. In some embodiments, the first conduit is an open channel and the second conduit is a lumen.

In some embodiments, the supporting splitter comprises a plurality of first and second conduits each shaped as an open conduit, circumferentially located near the walls of the body. In some embodiments, at least the first conduit is tilted at an angle of between 0° to 10° with respect to the central longitudinal axis of the body.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A, 1B, 1C, and 1D are illustrations of a device for multisite injection and it's components according to some embodiments of the invention;

FIG. 2 is an illustration of an injection unit according to some embodiments of the invention;

FIG. 3 is an illustration of another device for multisite injection according to some embodiments of the invention;

FIGS. 4A and 4B are flowcharts of methods for multisite injection according to some embodiments of the invention;

FIGS. 5A and 5B are illustrations of a supporting splitter according to some embodiments of the invention;

FIGS. 6A and 6B are illustrations of another supporting splitter according to some embodiments of the invention;

FIGS. 7A and 7B are illustrations of another supporting splitter according to some embodiments of the invention;

FIG. 8 is a graph showing tumor growth rate after treatment according to embodiments of the invention in comparison with conventional methods;

FIG. 9 shows tumor weight after treatment according to embodiments of the invention in comparison with conventional methods; and

FIG. 10 shows survival curves accoridng to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

### Devices for Multisite Injection

Some aspects of the invention are directed to devices for simultaneous multisite injection. Such devices may allow a controlled multisite injection of one or more types of fluids (e.g., medications or treatments) in a simple way.

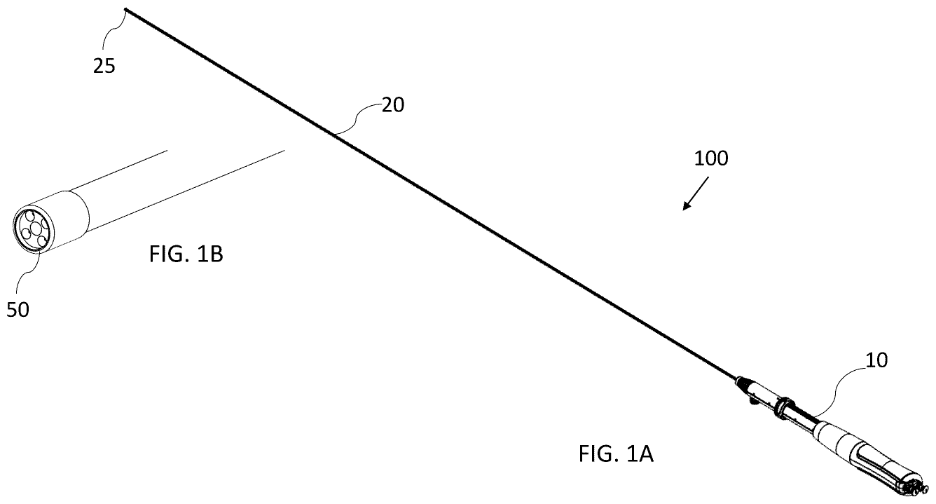

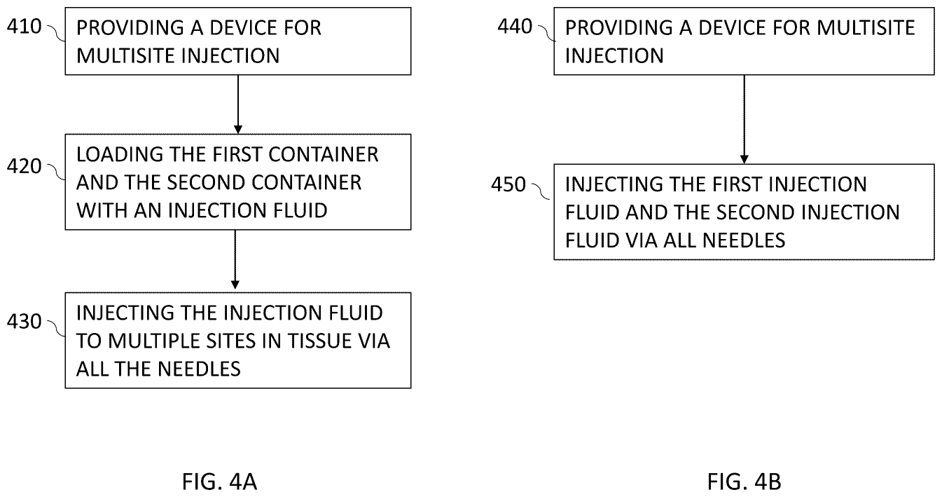

Reference is now made to FIGS. 1A, 1B, 1C, and 1D which are illustrations of a device for multisite injection and it's components according to some embodiments of the invention. A device 100 may include an injection unit 10 configured to inject an injection fluid via a plurality of needles 5. In some embodiments, all needles 5 may be held by a catheter 20. In some embodiments, catheter 20 comprises a plurality of lumens 21, each being in fluid connection with one needle 5. In some embodiments, device 100 may include a supporting splitter 50 located at a distal end 25 of catheter 20 opposite to injection unit 10. Some nonlimiting examples for supporting splitters 50 are given and discussed with respect to FIGS. 5 to 7 hereinbelow.

In some embodiments, the length of needles and catheter 20 may be between 200 to 2500 mm, for example, 200 to 1000 mm, 400 to 1400 mm, 500 to 1500 mm, 800 to 2000 mm, 1000 to 2400 mm and any value and range in between. In some embodiments, needles 5 may extend from a distal end of catheter 20 and may have a lateral spread or span of between 10 to 50 mm, for example, 10 to 20, 10 to 30, 10 to 40 mm and any value and range in between. In some embodiments, the length of the exaltation of needles 5 from catheter 20 may be between 50 to 120 mm, for example, 60 mm, 70 mm, 80 mm, 90 mm, 100 mm, 110, mm and any value in between. Therefore, device 100 may be configured to allow multisite injection at places in the body having limited direct accessibility, or during medical procedures that utilize catheters. In some embodiments, the very long catheters 20 and needles 5 may allow the user to enter the body from a cavity or an opening located more than 200 mm away (e.g., 1400 mm) from the treatment location, for example, for treating pancreatic cancer.

In some embodiments, the diameter of needle 5 may be between 18 to 26 Guage, for example, 20, 22, 24 and 25 Guage.

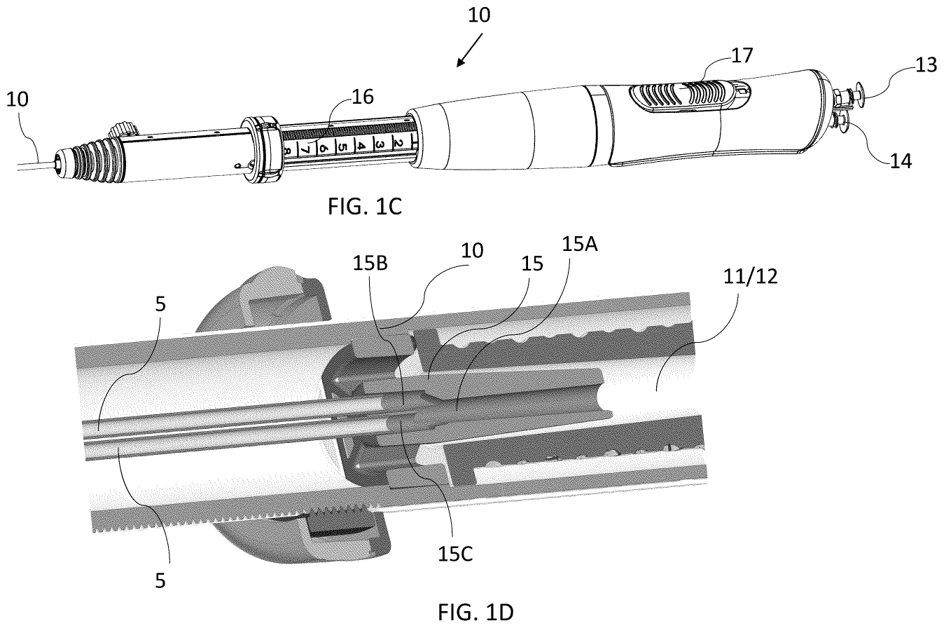

Device 100 may further include a first container 11 in fluid connection with at least two needles 5 and a second container 12 in fluid connection with at least one needle 5. An additional illustration of an injection unit and the containers is given in FIG. 2. In some embodiments, first container 11 and second container 12 are included in the same container. In some embodiments, two different containers 11 and 12 (shown in FIG. 2) may be included. In some embodiments, at least one of container 11 and/or container 12 may be connected to a divider 15 configured to divide the injection fluid between at least two needles 5. In some embodiments, divider 15 is included in catheter 20 or in injection unit 10. In some embodiments, divider 15 comprises a main lumen split 15A into two or more sub-lumens 15B and 15C, wherein each sub-lumen 15B and 15C is connectable to one needle 5. In some embodiments, divider 15 may evenly divide the amount of injection fluid held in container 11 or container 12 between at least two needles 5.

Accordingly, injection unit 10 is configured to inject a controlled amount of injection fluid from each needle, for example, the controlled amount is the same amount from at least two needles 5.

In some embodiments, first container 11 is configured to hold a first amount of injection fluid, and second container 12 is configured to hold a second amount of an injection fluid different from the first. In some embodiments, first container 11 is configured to hold a first type of injection fluid, and second container 12 is configured to hold a second type of injection fluid different from the first.

In some embodiments, injection unit 10 may further include at least two plungers 13 and 14. In some embodiments, plunger 13 may be configured to inject/pump injection fluid from first container 11, and plunger 14 may be configured to inject/pump injection fluid from second container 12. In some embodiments, injection unit 10 may further include a control button 17 and scale marks 16, indicating the amount of injection fluid in first and/or second containers 11 and/or 12. In some embodiments, control button 17 may control the movement of plungers 13 and/or 14 in order to provide the controlled amount of injection fluid.

In an alternative embodiment, injection unit 10 may include at least one pump (not illustrated) connected to first container 11 and/or second container 12. In some embodiments, the at least one pump may be configured to pump and/or inject a controlled amount of injection fluid from/to first container 11 and/or second container 12 into the corresponding needles 5. In some embodiments, two pumps may be included in injection unit 10, such that each pump is in fluid connection with one container. In some embodiments, a single pump may be in fluid connection to first container 11 and second container 12 via at least one switch configured to sequentially connect each container to the pump or connect both containers simultaneously to the pump.

In some embodiments, injection unit 10 is configured to inject the injection fluid simultaneously via all the needles 5, for example, by pushing simultaneously plungers 13 and 14, such that the injection fluid may flow simultaneously from both containers to all needles 5. In some embodiments, injection unit 10 is configured to inject the injection fluid sequentially by at least two needles, for example, by pushing plunger 13 first, following by pushing plunger 14, therefore, providing the injection fluid from first container 11 first to all corresponding needles 5 and then providing the injection fluid (same or different) from container 12 to all corresponding needles 5.

In some embodiments, device 100 may further include a controller (not illustrated) configured to control injection unit 10 to simultaneously or sequentially extract the injection fluid from the first container and the second container. For example, the controller may control the movement of plungers 13 and 14, using one or more actuators. In another example, the controller may control the at least one pump and or at least one switch and the like.

Reference is now made to FIG. 2 which is an illustration of another injection unit 10A to be included in device 100. Device 100 may include first container 11A and second container 11B. In some embodiments, first container 11A and the second container 11B are syringe barrels, and injection unit 10A is configured to control the movement of plungers 13 and 14 located in each syringe barrel 11A and 12A. In some embodiments, injection unit 10A may include divider 15 dividing the injection fluid held in container 11A between at least two needles 5. In some embodiments, catheter 20 may include at least two lumens 21 and 22 for directing needles 5. Lumen 21 may direct at least two needles 5 and lumen 22 may direct at least one needle 5, as illustrated.

In some embodiments, device 100 may be provided without the injection fluid and the user may fill the containers with the required injection fluid. In some embodiments, the user may use plungers 13 and/or 14 or the at least one pump to fill containers 11, 11A, 12, and/or 12A with one or more types of injection fluids. As should be understood by one skilled in the art, the two containers 11 and 12 or 11A and 12A are given as examples only, and device 100 may include more than two containers, more than three needles, more than one divider, and the like. In some embodiments, device 100 may be disposable or reusable. In some embodiments, device 100 may be a disposable device and the container may be prefilled with one or more types of injection fluid.

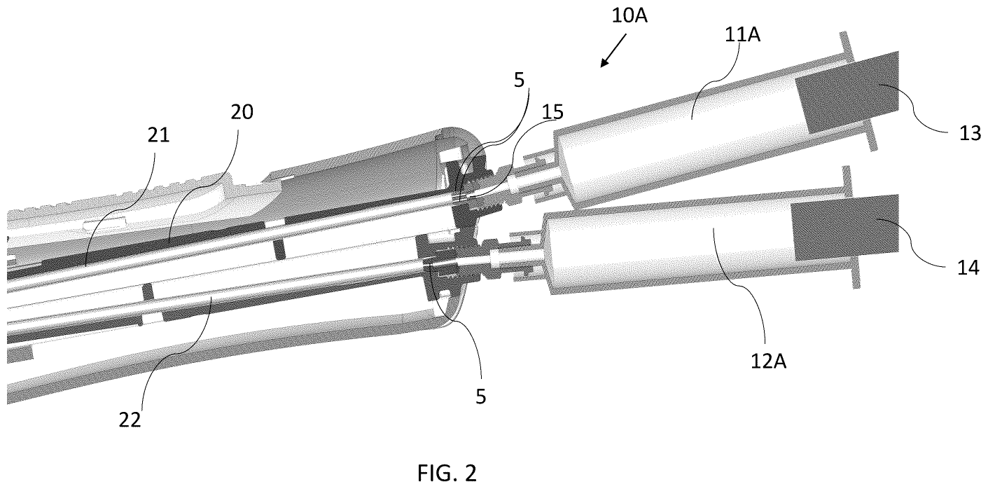

Reference is now made to FIG. 3 which is an illustration of another device for multisite injection according to some embodiments of the invention. In some embodiments, a device 200 may include prefilled one or more injection fluids 206 and/or 209, for example, for a single use. In some embodiments, device 200 may include a support 202 holding at least a first needle 204 in fluid connection to a first lumen 205 prefilled with a first injection fluid 206 and at least a second needle 207 in fluid connection to a second lumen 208 prefilled with a second injection fluid 209. Device 200 may further include an injection unit 210 configured to inject first injection fluid 206 and second injection fluid 209 via the corresponding needles 204 and 207.

In some embodiments, injection unit 210 may include at least one plunger 213 and/or a pump for injecting first injection fluid 206 and second injection fluid 209 from needles 204 and 207. In some embodiments, at least one plunger 213 and/or a pump is configured to apply pressure on first injection fluid 206 in first lumen 205 and on second injection fluid 209 in second lumen 208. In some embodiments, injection unit 210 may include unidirectional valves 215 configured to allow the flow of first injection fluid 206 and second injection fluid 209 only in a single direction towards the needles. In some embodiments, injection unit 10 is configured to inject the injection fluids simultaneously via all the needles 204 and 207, for example, when a single syringe barrel and plunger are included in device 200, as illustrated. In some embodiments, injection unit 210 is configured to inject injection fluids 206 and 209 sequentially by at least two needles 204 and 207. For example, injection unit 210 may include one syringe barrel with two plungers, or two syringe barrels and two plungers each being in fluid connection with one lumen 205 or 207, such that each plunger controls the provision of injection fluids 206 and 209 to corresponding needle 204 and 207.

In some embodiments, first injection fluid 206 and second injection fluid 209 are the same fluid. In some embodiments, first injection fluid 206 is provided in a first amount and second injection fluid 209 is provided in a second amount, different from the first. In some embodiments, the amounts of first injection fluid 206 and second injection fluid 209 are the same.

In some embodiments, injection unit 210 may include a pump (not illustrated) in addition or instead of syringe and plunger 213. The pump may be connected directly to each one of lumens 205 or 207 or via unit/syringe 210. The pump may allow injecting injection fluids 206 and 209 simultaneously or sequentially, by controlling the provision of an injection pressure to each one of lumens 205 or 207.

### Methods of Multisite Injection

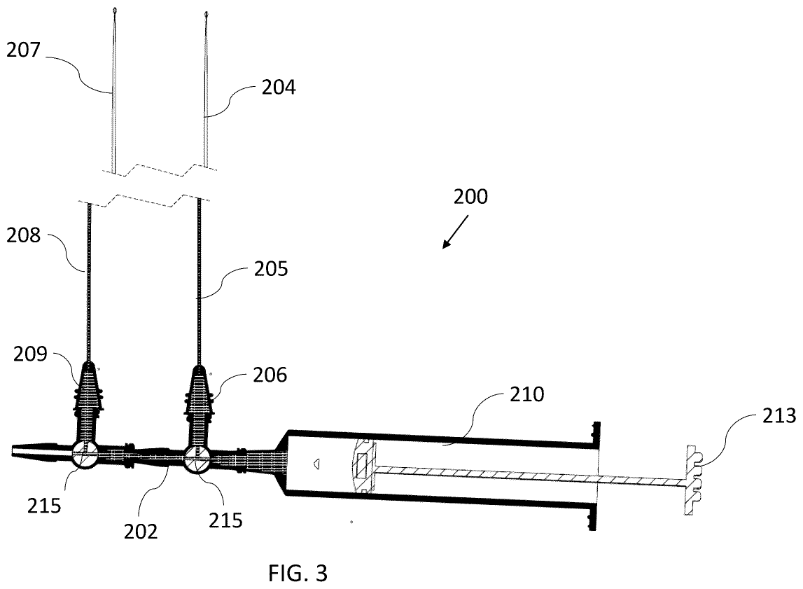

Reference is now made to FIG. 4A which is a flowchart of a method of multisite injection according to some embodiments of the invention. The method of FIG. 4A may be conducted using a multisite injection device 100. In step 410, device 100 may be provided, for example, to a user (e.g., a physician in an operation room, an emergency room, and a clinic, a researcher in a laboratory, and the like).

In step 420, first container 11 or 11A and second container 12 or 12A may be loaded with injection fluid. In some embodiments, the same injection fluid is loaded to first container 11 or 11A and second container 12 or 12A. In some embodiments, a first type of injection fluid is loaded to first container 11 or 11A and a second type of injection fluid is loaded into second container 12 or 12A.

In step 430, the injection fluid may be injected to multiple sites in tissue via all needles 5. In some embodiments, injecting the injection fluid is conducted situationally from all needles, for example, by pushing together plungers 13 and 14. In some embodiments, injecting the injection fluid is conducted sequentially by at least two needles, for example, by first pushing plunger 13 and then plunger 14. In some embodiments, injecting the injection fluid comprises injecting the same volume of injection fluid via at least two of the needles.

Reference is now made to FIG. 4B which is another method of multisite injection according to some embodiments of the invention. The method of FIG. 4B is conducted using device such as device 200 prefilled with one or more injection fluids. In step 440, a device such as, device 200 may be provided, for example, to a user (e.g., a physician in an operation room, an emergency room, and a clinic, a researcher in a laboratory, and the like). In step 450, first injection fluid 206 and second injection fluid 209 may be injected via all needles 204 and 207. In some embodiments, first injection fluid 206 and second injection fluid 209 may be the same fluid or two different fluids.

In some embodiments, injecting the first injection fluid and the second injection fluid is conducted situationally from all needles. In some embodiments, injecting the second injection fluid is conducted sequentially after the injection of the second injection fluid.

### Supporting Splitter

In some embodiments, in order to ensure that the needles (e.g., needles 5, 204, and 207) puncture the tissue in multiple distinct locations a splitter may be added at a distal end of a catheter included in any one of devices 100 or 200.

Reference is now made to FIGS. 5A and 5B which are a bottom view and cross-section view of a first non-limiting example of a supporting splitter according to some embodiments of the invention. A supporting splitter 50 for supporting needles may include a body 51 insertable into a catheter (such as catheter 20) holding the needles (e.g., needles 5, 204, and 207). Body 51 may include at least one first conduit 52 for supporting at least one first needle 5 into, and at least one second conduit 53 for supporting at least one second needle 5 such that the at least one first needle is spread from the at least one second needle. In some embodiments, at least one first conduit 52 is a first lumen and at least one second conduit 53 is a second lumen, as illustrated. In some embodiments, body 51 may include a plurality of first conduits 52 each shaped as a lumen, circumferentially located near the walls of body 51. In some embodiments, body 51 may include a plurality of first lumens 52 circumferentially located near the walls of body 51, and second conduit 53 which is a second lumen located along the longitudinal central axis of body 51, as illustrated. In some embodiments, lumens 52 of first conduits 51 are tilted at a predetermined angle of between 0° to 10° with respect to the central longitudinal axis of body 51, in order to align the needles in a required angle.

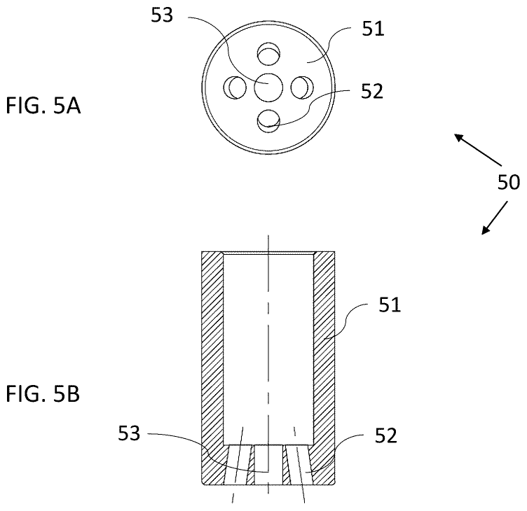

Reference is now made to FIGS. 6A and 6B which are perspective views of a second non-limiting example of a supporting splitter according to some embodiments of the invention. A supporting splitter 50A may include a body 51A insertable into a catheter holding the needles. In some embodiments, body 51A may include two portions ‘a’ and ‘b’. In some embodiments, portion ‘a’ may include one or more lumens for directing needles. In some embodiments, portion ‘b’ may include one or more first conduits shaped as open channels 54. In some embodiments, open channels 54 are tilted at a predetermined angle of between 0° to 10° with respect to the central longitudinal axis of body 51, in order to align the needles in the required angle. In some embodiments, the needles in open channels 54 are supported between open channels 54 and the inner walls of the catheter, for example, catheter 20. In some embodiments, supporting splitter 50A may further include at least one inner conduit 53 shaped as a lumen.

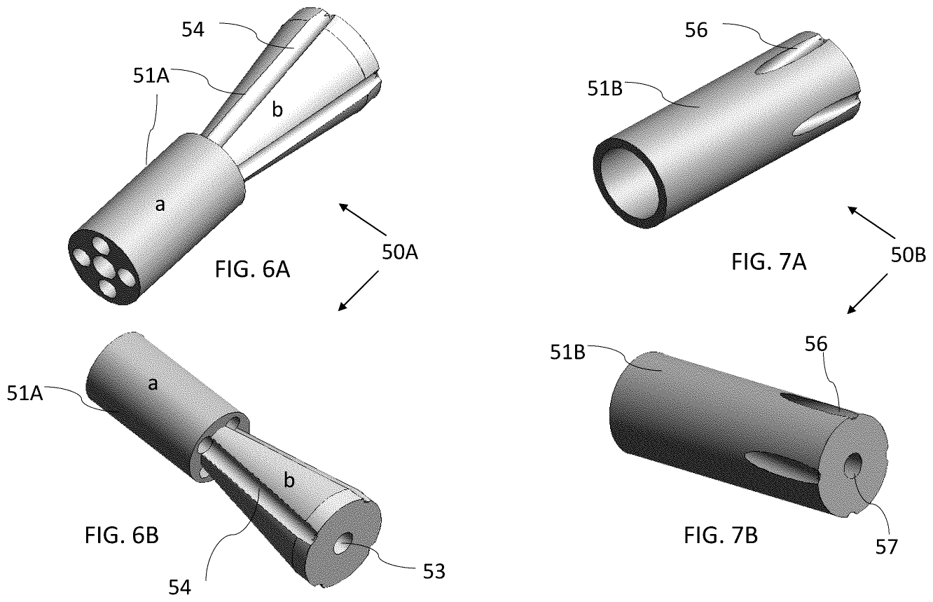

Reference is now made to FIGS. 7A and 7B which are perspective views of a second non-limiting example of a supporting splitter according to some embodiments of the invention. A supporting splitter 50B may include a body 51B insertable into a catheter holding the needles. In some embodiments, body 51B may be a hollow cylindrical body having at one end of the cylindrical body a base perorated with at least one hole 57. In some embodiments, one or more open channels 56 located on the outer walls of the cylindrical body may act as first conduits for directing a first group of needles. The needles in open channels 56 are supported between open channels 56 and the inner walls of the catheter, for example, catheter 20. At least one hole 57 may act as a second conduit.

### EXAMPLES

A very fine needle Hamilton-35 g was used for direct multisite injection of chemotherapy in combination with IV standard treatment of orthotopic pancreatic tumor and compared to single-site injection combined with IV treatment. Each group of subjects included 8 female nude mice; 6-8 weeks old. As should be understood by one skilled in the art, the experiments showing multi-site injection and treatment of cancer cells can be conducted in-vivo using device 100 accoridng to some embodiments of the invention.

The tumor used in this experiment was formed by making a knot at 2 mm from the end of the pancreas tail and 1×10⁵ cells of PANC1 cells (ATCC) were slowly injected at P7-8 using a 27 G needle. Treatment was initiated after 20 days, where the tumors had reached a median size of 50 mm³ (range 40-60 mm³). Table 1 summarizes the treatment given to each group.

TABLE 1

| | | Local administration/Total | # animal/ |
|---|---|---|---|
| Group # | IV Dose/Treatment | dose/Treatment | group |
| 1-Control | Saline | Saline | 8 |
| 2-L4O | Saline | oxaliplatin 250 ng | 8 |
| | | (divided to 4 sites) | |
| 3-IV | FOLFIRINOX (iv | Saline | 8 |
| | dose see below) | | |
| 4-L1O-IV | FOLFIRINOX | oxaliplatin 250 ng (single | 8 |
| | | spot of injection) | |
| 5-LAO-IV | FOLFIRINOX | oxaliplatin 250 ng | 8 |
| | | (divided to 4 sites) | |

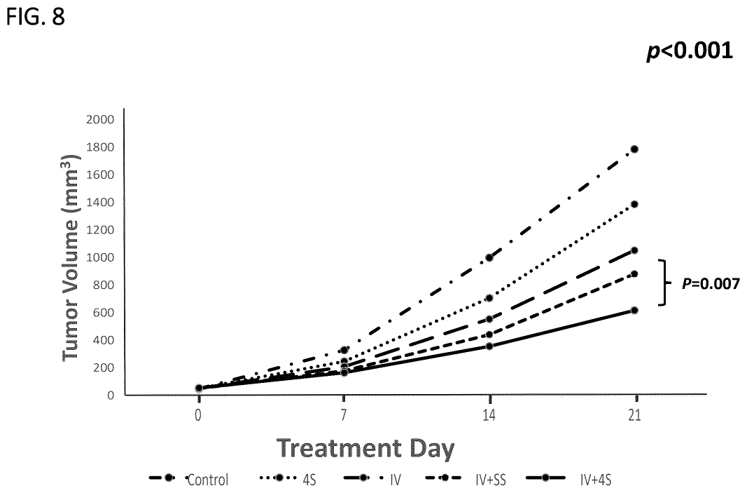

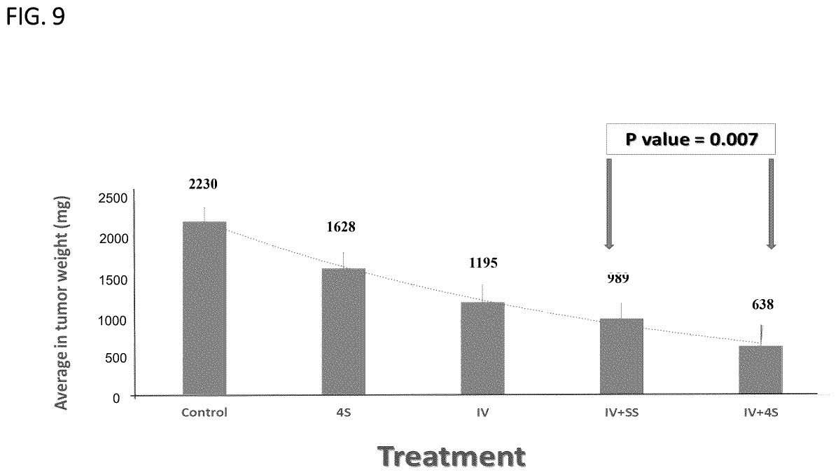

All subjects were monitored twice a week for, weight, general signs of morbidity and immobility (scale of 1-5) and survival/mortality. The tumor volume (mm³) as a measure to growth rate, and tumor weight (mg) were measured during and after the end of treatment, respectively. In the end, the following tests were conducted, necroscopy and histopathological examination. Three treatment cycles were given to each group. Tumor volume was measured prior to each treatment and at sacrifice day. Results are plotted and are presented as the growth rate by time (FIG. 8). The excised tumors at the end of the experiment were weighted and the results are shown in FIG. 9. Group 5 (L4O-IV) which was provided with a combination of FOLFIRINOX and 4 multisite injections of oxaliplatin 250 ng showed a larger decrease in tumor size, as shown in FIGS. 8 & 9. Injecting the same dose using a single injection group 4 (L1O-IV) showed about 40% less decrease in tumor rate of growth and size, demonstrating the benefits of using a device such as device 100 in tumor treatments.

Another experiment was done to monitor survival, using the direct multisite injection (4S+IV) of chemotherapy according to some embodiments of the invention compared to treatment with saline (control), one site injection (SS+IV), and conventional treatment used as a standard of care (IV). In groups 4S+IV and SS+IV the animal were a augmented with IV treatment.

After 4 weeks of combination therapy, the animals were treated IV only up to death. Subsequent analysis (Kaplan Meier) for their median survival indicated that Median survival of the multisite+IV group was 60 days, compared to 54, 49 and 41 days of the one-site+IV, IV and control groups, respectively (p=0.004, p<0.0001, p<0.0001, respectively). In multiple comparison, the adjusted p values are given in table 2

TABLE 2

| | Adjusted p- |
|---|---|
| | value(Dunn- |
| Comparisons | Sidak) |
| control vs IV | 0.003 |
| control vs ss + iv | <0.0001 |
| control vs 4s + iv | <0.0001 |
| IV vs ss + iv | 0.030 |
| IV vs 4s + iv | <0.0001 |
| ss + iv vs 4s + iv | 0.004 |

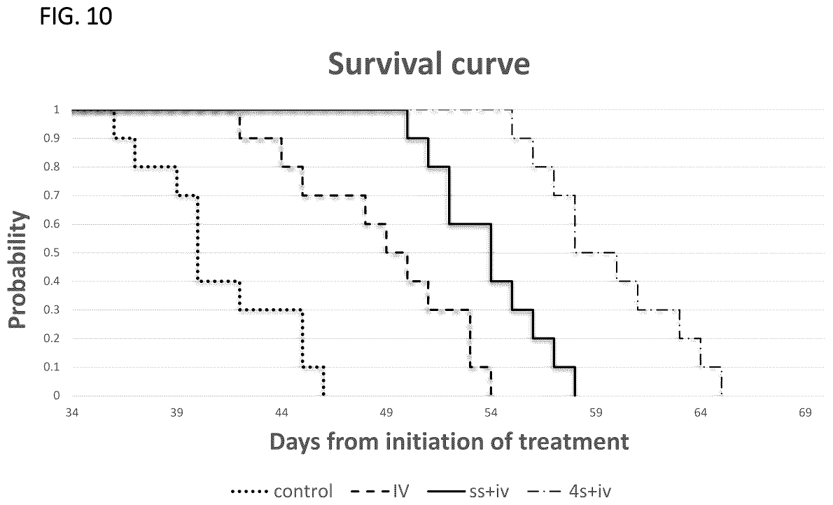

The survival curve is shown in FIG. 10.

As clearly shown from all the compared results, the use of the multisite injection device and method accoridng to embodiments of the invention has great benefits in making conventional medications used for treating the tumor to be much more effective. The experiments showed both significant inhibition in tumor growth rate and size, and in increase in survival time of the animals bearing human orthotopic tumor.

Unless explicitly stated, the method embodiments described herein are not constrained to a particular order or sequence. Furthermore, all formulas described herein are intended as examples only and other or different formulas or active substance may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A device for multisite injection, comprising:
a first container in fluid connection with at least two needles;
a second container in fluid connection with at least one needle; and
an injection unit configured to inject an injection fluid from the first container and the second container via all the needles.

2. The device of claim 1, wherein the injection unit is configured to inject the injection fluid simultaneously via all the needles.

3. The device of claim 1, wherein the injection unit is configured to inject the injection fluid sequentially by at least two needles.

4. The device according to any one of claim 1, wherein the injection unit is configured to inject a controlled amount of injection fluid from each needle.

5. The device of claim 4, wherein the controlled amount is the same amount from at least two needles.

6. The device according to claim 1, further comprising a catheter comprising a plurality of lumens, each being in fluid connection with one needle.

7. The device of claim 6, further comprising a supporting splitter located at a distal end of the catheter opposite to the injection unit, wherein the supporting splitter comprises a plurality of conduits for directing each one of the needles.

8. The device according to any one of claim 1, wherein the first container and the at least two needles are connected via a divider configured to divide the injection fluid between the at least two needles.

9. The device of claim 8, wherein the divider is included in a catheter.

10. The device of claim 9, wherein the divider comprises a main lumen split into two or more sub-lumens, wherein each sub-lumen is connectable to one needle.

11. The device according to any claim 1, wherein the first container is configured to hold a first amount of injection fluid and the second container is configured to hold a second amount of injection fluid different from the first.

12. The device according to claim 1, wherein the first container and the second container are syringe barrels and wherein the injection unit is configured to control the movement of a plunger located in each syringe barrel.

13. The device according to claim 1, wherein the injection unit comprises at least one pump connected to at least one of the first container and the second container.

14. The device according to claim 1, further comprising a controller configured to control the injection unit to simultaneously extract the injection fluid from the first container and the second container.

15. (canceled)

16. (canceled)

17. (canceled)

18. (canceled)

19. (canceled)

20. (canceled)

21. (canceled)

22. (canceled)

23. A method of multisite injection, comprising:
providing a device for multisite injection, comprising:
a first container in fluid connection with at least two needles;
a second container in fluid connection with at least one needle; and
an injection unit configured to inject an injection fluid from the first container and the second container via all the needles;
loading the first container and the second container with an injection fluid; and
injecting the injection fluid to multiple sites in a tissue via all the needles.

24. The method of claim 23, wherein injecting the injection fluid is conducted situationally from all needles.

25. The method of claim 23, wherein injecting the injection fluid is conducted sequentially by at least two needles.

26. The method according to claim 23, wherein injecting the injection fluid comprises injecting the same volume of injection fluid via at least two of the needles.

27-37. (canceled)
